(19) United States
(12) Patent Application Publication
(10) Pub. No.: US 2006/0251698 A1
(43) Pub. Date: Nov. 9, 2006

(54) Device for axon and neurite growth and method for producing the same

(75) Inventors: Hsin-Hsin Shen, Hsinchu County (TW); Wan-Shiun Lou, Taichung City (TW); Chi-Hung Lin, Taipei City (TW); Hong-Wen Liu, Taipei City (TW)

Correspondence Address: BACON & THOMAS, PLLC, 625 SLATERS LANE, FOURTH FLOOR, ALEXANDRIA, VA, 22314, US

(73) Assignee: Industrial Technology Research Institute, Hsin Chu (TW)

(21) Appl. No.: 11/246,243

(22) Filed: Oct. 11, 2005

(30) Foreign Application Priority Data
May 4, 2005 (TW) 94114379

(51) Int. Cl. A61F 2/06

(52) U.S. Cl. 424/423; 623/1.11

(57) ABSTRACT

The present invention relates to a device for directing and accelerating the growth of neuronal axon or neurite. The invention accelerates the growth speed of axon of nerve cells by using a substrate configured with a discontinuous micropattern of neuronal path-finding molecules or material thereon. The invention can reduce the connection or regeneration time of neural network. The present invention also discloses a micro-contact printing method for producing the device.

### BACKGROUND OF THE INVENTION

1. Field of the Invention

The present invention discloses a device for directing and accelerating the growth of neuronal axon and method for producing the same, which may be applied in the culture of nerve cells and the regeneration of nerve tissues.

2. Description of Related Art

The senses, movements, thinking, and emotions of human body and even the functioning of organs are integrated and regulated by the nervous system. Damage or pathological changes to any parts of the nervous system can cause great inconvenience to the life of the patient, and at times adversely affect the consciousness and mental state of patient. Thus any technology or product that can help maintain the normal working of the nervous system has its importance and urgency.

The physiological functions of the nervous system are performed by nerve cells or what we call “neurons.” Structurally, a neuron comprises two parts—soma and processes. The processes further differentiate to axon and dendrite. The axon transfers message to another neuron, while dendrites receive the message transmitted by another neuron. At the distal end of processes there is a bulging structure called synapse. Synapse is the contact between two neurons through which the neurotransmitter secreted by one neuron is relayed to the other neuron. Similarly the other neuron receives the stimulus of neurotransmitter through synapse to trigger a succession of message relay actions and reactions in cell bodies.

There are two requirements for a mature nervous system to maintain its basic functions: first neurons must be located at specific sites, and the connections of neuronal processes (axon and dendrite) or the synapses must be accurate. The locations of neurons and their connection targets are not randomly generated, but through a series of differentiation and developmental processes. In the process of development, individual nerve cells migrate along different paths to settle down at specific locations. Besides cell location, the connection between nerve cells must be accurate, which relies on the growth cone at axonal terminal. The growth cone extends along a specific path to finally contact its target before it differentiates into synapse. Different growth cones have distinct extension path. Morphologically a typical growth cone has some microspikes supported by actin. Growth cones rely on the elongation or shortening of microspikes to advance forward or change direction.

The extension of axonal growth cone not only plays a vital role in growth and development. When nerve tissue is damaged, the search for regeneration pathway and target connection by axonal growth cone are keys to whether the damaged nerve can restore functions. Thus if the mechanisms for axonal extension (or regeneration) and the path-finding of growth cone are fully understood, such knowledge can be applied in nerve tissue engineering to help the development of related products, such as the pattern design of nerve regeneration guide or the invention of neuronal path-finding molecules. Similar knowledge about the growth characteristics of axon and growth cone may be applied in the development of drugs with neuroprotective or neurotrophic activities.

The extension mechanism of axonal growth cone has been an important research subject in neural science. Literature shows that a considerable number of scientists have attempted to apply the material science and Micro-Electro-Mechanical Systems (MEMS) technologies to in-vitro culture of nerve cells to guide the axon to grow in a specific direction and result in a specific pattern of neural network. Such experiments also facilitate the recording of axonal growth parameters, the analysis of cell membrane potential and the secretion of neurotransmitter. Max Planck Institute of Germany is probably the organization that has invested most heavily in this kind of research and achieved the most substantial results. Its technological development is primarily directed at: 1. fabrication of patterned silicon chip; 2. surface modification with neuronal path-finding molecule; and 3. in-vitro culture of nerve tissues or cells on material to form neural network. The technologies developed by Max Planck Institute however only offers the basic framework of neural network. There remain many bottlenecks in the in-vitro construction of neural network. For example, 1. How to find a breakthrough in the contact between nerve cell and material interface that allows full reception of transmitted message and relay of stimulus; 2. How to prolong the survival of nerve cells cultured on substrate to enhance their use value; 3. How to construct a real-time axonal growth detection system to gain insight into the mechanism of axonal extension; 4. How to design neural network chips for use in the screening of neural drugs, guiding the regeneration of nerve tissues, and transfer/reception of neural messages; and 5. How to more effectively direct axonal growth along a certain pathway?

In the field of culture and regeneration of nerve cell, US Patent No. 20030032946 discloses a technology that uses 50-100 nm wide channels to guide axonal connection which may be applied in the intra-tissue connection of neural network. In the paper published by L. Lauer et al. 2002 entitled “Electrophysiological recordings of patterned rat brain stem slice neurons”, there discloses the design of patterns with different line and node sizes (line size of 2˜6 microns and node size of 10˜20 micron), where protein is coated on the patterned culture substrate. But the growth-guiding molecules in the aforesaid technology provide a continuous path or the substrate has heavier coating of path-finding molecules at specific modes, which apparently differs from the discontinuous pattern of path-finding molecules disclosed in this invention.

Conventional nerve cell culture devices or methods typically adopt the design of continuous guide path or alter the materials coated on substrate, and take longer for nerve cells to grow. Thus it is essential to develop a device that can accelerate the regeneration and outgrowth of nerve cells.

### SUMMARY OF THE INVENTION

To effectively promote the growth and regeneration of nerve cells and control the axonal extension, the present invention uses a substrate configured with a micropattern to culture nerve cells or direct nerve regeneration, accelerate the speed of axonal extension, and reduce the connection or regeneration time of neural network.

The object of the present invention is to provide a device for axon or neurite growth, comprising a substrate having a surface that allows the adhesion of nerve cells thereon; and path-finding molecules disposed on the surface of substrate to direct the growth of neuronal axon; wherein the distribution of path-finding molecules is at least in part discontinuous, preferably in a pattern of discontinuous lines or discontinuous blocks with 2˜10 micron wide and a space of 2˜15 micron apart for every segment of 5˜100 micron. The substrate can further contain a neuron cultivation area.

Another object of the present invention is to provide a method for producing a device for axon or neurite growth, comprising the steps of: (a) providing a microcontact printing stamp having discontinuous pattern thereon; (b) forming path-finding molecules on the microcontact printing stamp; (c) laying the microcontact printing stamp in step (b) over a substrate to transfer the path-finding molecules onto the substrate; and (d) separating the microcontact printing stamp from the substrate to obtain a device with path-finding molecules to direct neurite and axon growth, and the distribution of path-finding molecules on the device is at least in part discontinuous.

Yet another object of the present invention is to provide a method for culturing neurons using the aforesaid device for axon or neurite growth, comprising the steps of: providing neurons to be cultured; placing the neurons on the path-finding molecules of the device for axonal or neurite growth according to the present invention; and placing the device in an environment suitable for culture of neurongrowth to induce growth of axon or neurite.

In the device for axon or neurite growth, the path-finding molecules are distributed in an interrupted or discontinuous pattern, which, when used in neuron culture or nerve regeneration, can control the direction of axonal growth and accelerate axonal extension. Thus the device may be applied in the culture of neuron, fabrication of embedded neuronal chip, construction of in-vitro neural network, neural regeneration substrate, and scaffolding for repair of nerve tissue.

### BRIEF DESCRIPTION OF THE DRAWINGS

FIG. 1A shows a microcontact printing stamp having the pattern of straight lines and round cell cultivation areas.

FIG. 1B shows a microcontact printing stamp having the pattern of straight lines and interrupted lines.

FIG. 1C shows the SEM image of the surface of microcontact printing stamp having he pattern of interrupted lines.

FIG. 2A shows the device for axonal or neurite growth having a discontinuous pattern of path-finding molecules according to the invention, where the discontinuous pattern of path-finding molecules features a 5 μm gap for every segment of 10 μm.

FIG. 2B shows the device for axonal or neurite growth having a discontinuous pattern of path-finding molecules according to the invention, where the discontinuous pattern of path-finding molecules features a 10 μm gap for every segment of 100 μm.

FIG. 2C shows the conventional path-finding molecules having a continuous pattern.

FIG. 3A shows the chart of axonal extension of a neuron cultured on the device according to the invention vs. time, where the discontinuous pattern of path-finding molecules features a 5 μm gap for every segment of 50 μm.

FIG. 3B shows a bar chart comparing the average speed of axonal growth on path-finding molecules having discontinuous and continuous pattern.

FIG. 4A shows the chart of axonal extension of a neuron cultured on the device according to the invention vs. time, where the discontinuous pattern of path-finding molecules features a 5 μm gap for every segment of 20 μm.

FIG. 4B shows a bar chart comparing the average speed of axonal growth on path-finding molecules having discontinuous and continuous pattern.

FIG. 5A shows the chart of axonal extension of a neuron cultured on the device according to the invention vs. time, where the discontinuous pattern of path-finding molecules features a 10 μm gap for every segment of 40 μm.

FIG. 5B compares the average speed of axonal growth on path-finding molecules having discontinuous and continuous pattern.

### DETAILED DESCRIPTION OF THE INVENTION

The device for axon or neurite growth according to the invention comprises a substrate and path-finding molecules. The substrate may be a tissue scaffold, nerve regeneration guiding device, culture dish, chip or microfluidic chip made of silicon, glass, quartz, polymer or culture medium. The substrate has a surface that allows adhesion and growth of neurons thereon. The growth of neuronal axon along a predetermined direction relies on the action of path-finding molecules. For the path-finding molecules to form more steadfastly on substrate surface, recessed or protruded lines or blocks may be configured on substrate surface in one body or etched or deposited on the surface during substrate production to facilitate the formation of path-finding molecules. The substrate may also be a commonly known nerve regeneration guiding device or scaffold for nerve tissue repair, which, through the special distribution pattern of path-finding molecules according to the invention, enhances the growth and regeneration of neuron on the substrate.

The path-finding molecules configured on the substrate surface to direct the growth of neuronal axon may be protein, peptide, neurotransmitter, biocompatible composite material, their mixtures, and other substances that aid the growth, regeneration and adhesion of nerve cells. The path-finding molecules may be bound to substrate surface using prior art, including chemical immobilization, coating, jet spraying, and micro-contact printing.

The pattern of path-finding molecules is interrupted lines or discontinuous blocks, preferably discontinuous lines 2˜10 μm wide with a 2˜15 μm gap for every segment of 20˜100 μm.

The substrate surface of the device for neuronal growth may further include chemical attractant, adhesion molecule, repulsive molecule and surface contour material known in the field for controlling and promoting neuronal growth and regeneration as well as axonal extension to facilitate the extension of axon towards a predetermined direction.

One of the methods for preparing the device for neuronal growth according to the invention is to use microcontact printing method, in which, a microcontact printing stamp is first fabricated by making a master mold by machine cutting, etching or photomask lithography, followed by the casting process. The microcontact printing stamp is made of soft polymer material, such as polydimethyl siloxane (PDMS), polystyrene (PS), polypropylene (PP), or mixture thereof, preferably PDMS. The steps for fabricating the microcontact printing stamp are as follows: deposit the stamp material in fluid state on a flat-surface material (e.g. glass slide), lay the flat-surface material over the mold, place the mold and flat-surface material in a vacuum system until the air between flat-surface material and mold is completely eliminated, then bake the die in high-temperature until the soft polymer material is cured before removing it from the die. Consequently, the micro-contact printing stamp will have a pattern complementary to the mold. In this invention, the design that will form discontinuous pattern of path-finding molecules will be used.

After the microcontact printing stamp is made, path-finding molecules are formed on it, during which, nitrogen spray gun may be used to accelerate the drying and formation of path-finding molecules. Subsequently, a substrate onto which path-finding molecules will be printed is laid over the microcontact printing stamp. The substrate may first be acid washed and rinsed with water before soaking in alcohol, and then baked before use. In addition, prior to the formation and transfer of path-finding molecules, the substrate and microcontact printing stamp may be surface treated with O₂ plasma to render it more hydrophilic to facilitate the adhesion of path-finding molecules.

The substrate and the microcontact printing stamp are pulled part using a blade after the transfer of path-finding molecules to facilitate the separation and prevent twice overlapping of the substrate and microcontact printing stamp.

The method for preparing a device for axon or neurite growth with discontinuous neuronal path-finding molecules is not limited to microcontact printing just described. Any method that enables the neuronal path-finding molecules to adhere to the substrate may be used. The embodiments of the device according to the invention include culture dish, neuronal chip, microfluidic chip, nerve regeneration conduit, and scaffold for repair of nerve tissue, which may be applied to neuron culture, fabrication of embedded neuronal chip, construction of in-vitro neural network, and neural regeneration substrate.

The advantages of the present invention are further depicted with the illustration of examples, which however should not be construed as a limitation on the scope of claim.

### EXAMPLE 1

### Preparing the Device for Axon or Neurite Growth

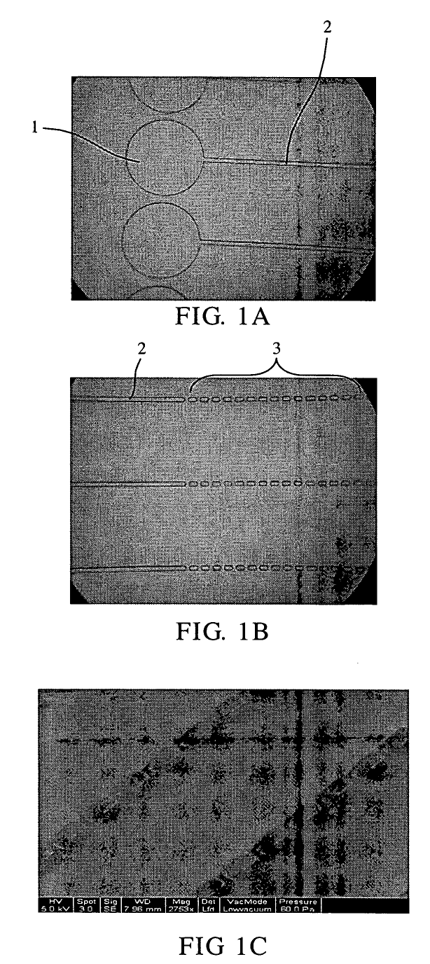

First produce a silicon mold using lithography and prepare a mixture of Sylgard silicone elastomer 184 and PDMS in 10:1 ratio. Take a slide and place the prepared PDSM solution on it. Lay the slide over the mold and eliminate the air between the slide and the mold with a vacuum pump to until all airpumped out. Bake the mold in 70° C. environment and remove PDMS from the die after it is cured to obtain a PDMS stamp (as shown in FIG. 1A˜1C). From FIG. 1A and FIG. 1B, the pattern of microcontact printing stamp in this example features round neuron cultivation areas 1, straight lines 2 (FIG. 1A), and interrupted lines 3 immediately behind the end of straight lines 2. Such topographic structure is for printing onto the substrate a cell cultivation area for placement and culturing of neurons, while the straight line configuration 2 and interrupted line configuration 3 are for printing onto the substrate discontinuous pattern of path-finding molecules to direct and accelerate axonal growth.

Next take another slide as substrate. The slide is washed, rinsed with water, soaked in alcohol and baked before use. Subject the slide and microcontact printing stamp to surface treatment with O₂ plasma, and then deposit laminin as path-finding molecules onto the printing stamp and blow it with nitrogen gun to allow even distribution of molecules on stamp surface. After the path-finding molecules are dried, lay the slide over printing stamp to proceed with the transfer. Gently press the slide in the process, but over-exertion of force should be avoided to prevent blurry print. Subsequently separate the slide from the stamp with the aid of a blade if necessary. Avoid twice overlapping the slide and the stamp in the process. After separation, a device for the growth of neuronal axon or neurite is obtained.

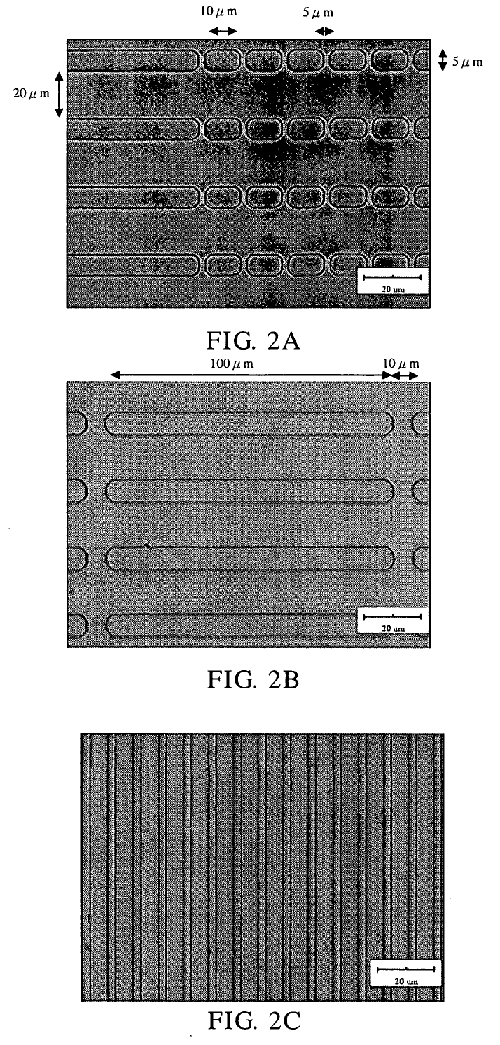

The device for the growth of neuronal axon or neurite having discontinuous pattern of path-finding molecules produced by microcontact printing as shown in FIG. 2A and FIG. 2B is apparently different from the conventional device having continuous pattern of path-finding molecules as shown in FIG. 2C. The lines of path-finding molecules in FIG. 2A are 5 μm wide with 20 μm distance between two parallel lines and each line is arrayed in a repetitive pattern of every 10 μm (dash line: 10 μm) of straight segment followed by a 5 μm gap (dash gap: 5 μm). In FIG. 2B, the width of the line and distance between two parallel lines are also 5 μm and 20 μm respectively, but each line is arrayed in a repetitive pattern of every 100 μm of straight segment followed by a 10 μm gap. In FIG. 2C the line width of path-finding molecules is 3 μm and the distance between two parallel lines is 6 μm.

### EXAMPLE 2

### Using the Device for Axon or Neurite Growth According to the Invention to Culture Goldfish Neurons

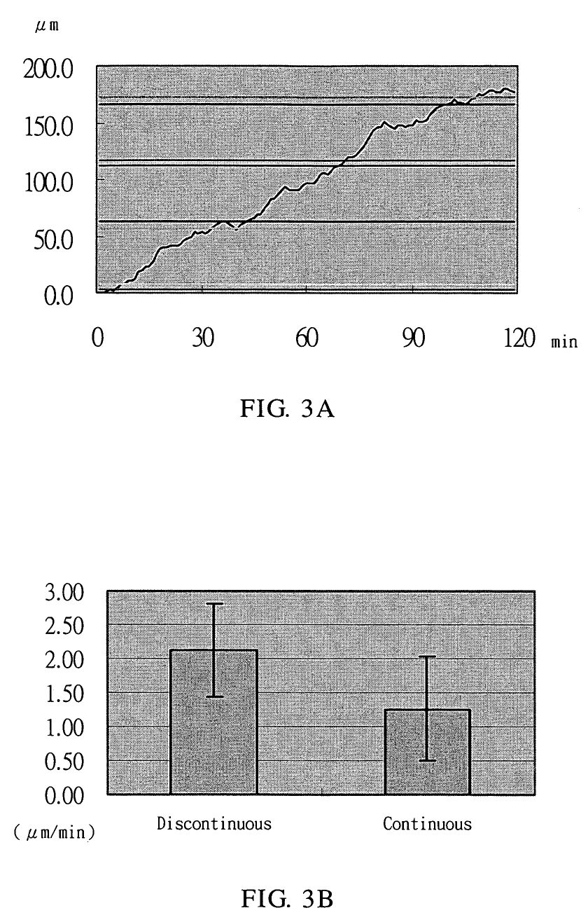

Goldfish retinal ganglion tissue mass are placed on the path-finding molecules of the device for axon or neurite growth according to the invention. The substrate in this example is regular slide, and the path-finding molecules are laminin with a pattern of interrupted lines having a 5 μm gap for every segment of 50 μm. The control group has a pattern of continuous lines as shown in FIG. 2C. The device carrying the goldfish ganglion cells is placed in a proper environment to observe the growth of axon and neurite. The results are as shown in FIG. 3A and FIG. 3B. In FIG. 3A which shows the chart of axonal extension of ganglion cell cultured on the device according to the invention vs. time, we can see that axon was successfully directed and extended in the first 120 minutes. FIG. 3B compares the effect of discontinuous and continuous pattern of path-finding molecules on the speed of axonal extension. From the bar chart of average speed, it is learned that the discontinuous pattern of path-finding molecules induced the growth of axon at nearly twice the speed as the continuous pattern. Its mechanism could be that growth cone leaps across the gap interface of path-finding molecules, thereby accelerating the axonal extension.

### EXAMPLE 3

### Using the Device for Axon or Neurite Growth According to the Invention to Culture Goldfish Neurons

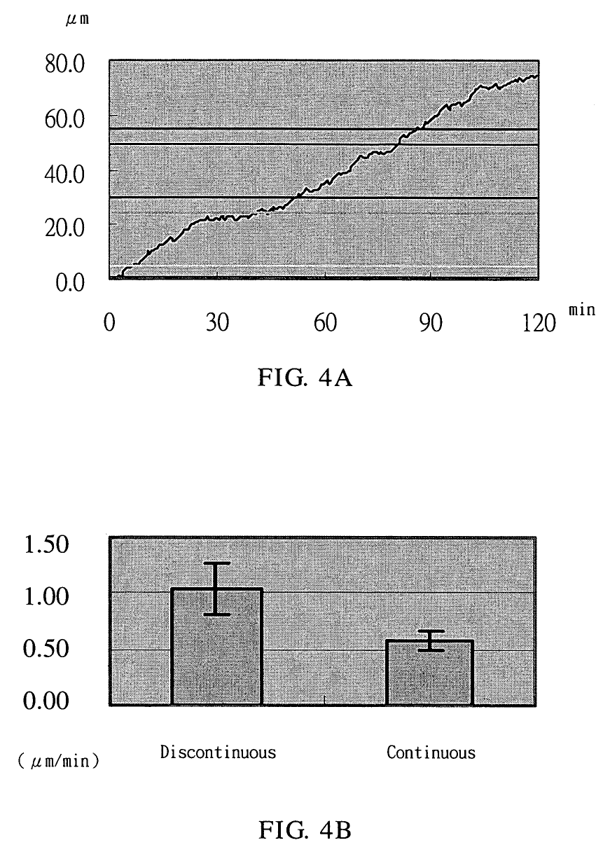

The preparations and experimental steps in this example are the same as Example 2, only the pattern of path-finding molecular material features a 5 μm gap for every segment of 50 μm. The control group similarly has a pattern of continuous lines. The results are as shown in FIG. 4A and FIG. 4B. In FIG. 4A which shows the chart of axonal extension of ganglion cell cultured on the device according to the invention vs. time, we can see that axon was successfully directed and extended in the first 120 minutes. FIG. 4B compares the effect of discontinuous and continuous pattern of path-finding molecules on the speed of axonal extension. From the bar chart of average speed, it is learned that the discontinuous pattern of path-finding molecules induced the growth of axon at nearly twice the speed as the continuous pattern.

### EXAMPLE 4

### Using the Device for Axon or Neurite Growth According to the Invention to Culture Goldfish Neurons

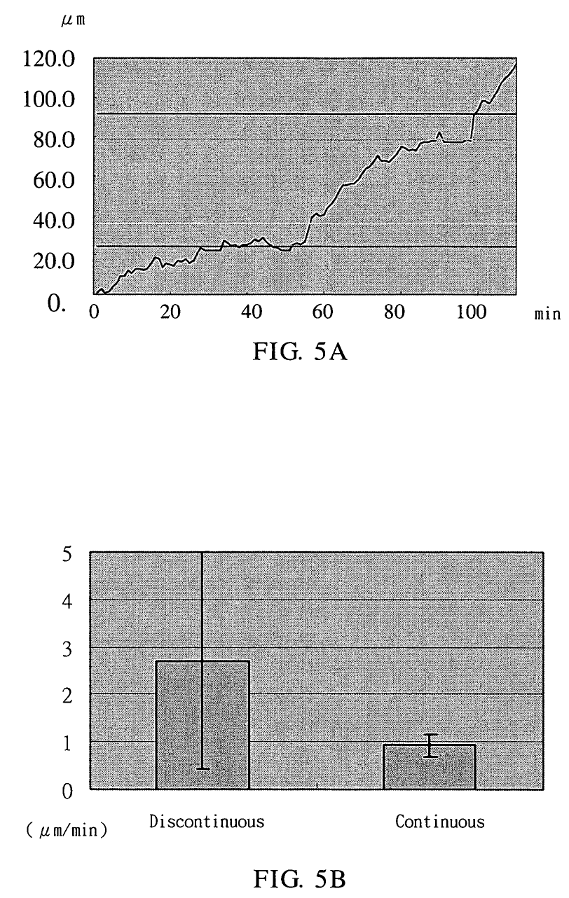

The preparations and experimental steps in this example are the same as Example 2, only the pattern of path-finding molecular material features a 10 μm gap for every segment of 40 μm. The control group similarly has a pattern of continuous lines. The results are as shown in FIG. 5A and FIG. 5B. In FIG. 5A which shows the chart of axonal extension of ganglion cell cultured on the device according to the invention vs. time, we can see that axon was successfully directed and extended in the first 120 minutes. FIG. 5B compares the effect of discontinuous and continuous pattern of path-finding molecules on the speed of axonal extension. From the bar chart of average speed, it is learned that the discontinuous pattern of path-finding molecules induced the growth of axon at nearly 2.5 times the speed as the continuous pattern.

Based on the results of the examples shown above, when applied to neuron culture or nerve regeneration, the device for axon or neurite growth having discontinuous pattern of path-finding molecules disclosed in the present invention can promote the neurite growth on substrate surface, guide the direction of axonal growth and accelerate the speed of axonal extension. In comparison with prior devices for directing the growth of neuronal axon or neurite, the present invention apparently increases the speed of neurite extension, which presents a major breakthrough in the science of nerve reconstruction and regeneration treatment.

### Other Embodiments

A few embodiments of the invention have been disclosed. All modifications and alterations to the descriptions disclosed made by those familiar with the skill without departing from the spirits of the invention and appended claims shall remain within the protected scope and claims of the invention.

What is claimed is:

1. A device for axon or neurite growth, comprising:
a substrate having a surface that allows the adhesion and growth of nerve cells thereon; and
path-finding molecules configured on said surface of the substrate to direct the growth of neuronal axon;
wherein a distribution of said path-finding molecules is at least in part in discontinuous pattern.

2. The device according to claim 1, wherein the distribution of said path-finding molecules has a pattern of discontinuous lines.

3. The device according to claim 1, wherein the distribution of said path-finding molecules has a pattern of discontinuous blocks.

4. The device according to claim 2, wherein the discontinuous lines are 2˜10 micron wide.

5. The device according to claim 2, wherein the discontinuous lines have a 2˜15 micron gap for every segment of 5˜100 micron.

6. The device according to claim 1, wherein the path-finding molecules include protein, peptide, neurotransmitter, biocompatible composite material or their mixtures thereof.

7. The device according to claim 1, wherein the surface of the substrate can further be first configured with an axonal guide path in discontinuous pattern by means of etching or deposition for the coating of path-finding molecules thereon.

8. The device according to claim 1, wherein the path-finding molecules can be attached to the substrate surface by means of chemical immobilization, coating, jet spray or microcontact printing.

9. The device according to claim 1, wherein the substrate can further include a nerve cell cultivation area.

10. The device according to claim 1, wherein the surface of substrate can further contain chemical attractant, adhesion molecule, repulsive molecule and surface contour material.

11. A method for preparing a device for axon or neurite growth, comprising the steps of:
(a) providing a microcontact printing stamp having a discontinuous pattern thereon;
(b) forming path-finding molecules on the microcontact printing stamp;
(c) laying the microcontact printing stamp in step (b) on a substrate to transfer the path-finding molecules onto the substrate; and
(d) separating the microcontact printing stamp and the substrate to obtain a device configured with path-finding molecules to direct the growth of neuronal axon or neurite and the distribution of said path-finding molecules has at least in part a discontinuous pattern.

12. The method according to claim 11, wherein the microcontact printing stamp is produced by casting against a mold made by lithography.

13. The method according to claim 11, wherein the microcontact printing stamp is made of polydimethyl siloxane (PDMS), polystyrene (PS), polypropylene (PP) or mixtures thereof.

14. The method according to claim 13, wherein the microcontact printing stamp is made of polydimethyl siloxane (PDMS).

15. The method according to claim 11, wherein the distribution of the path-finding molecules has a pattern of discontinuous lines.

16. The method according to claim 11, wherein the distribution of the path-finding molecules has a pattern of discontinuous blocks.

17. The method according to claim 15, wherein the discontinuous lines are 2˜10 micron wide.

18. The method according to claim 15, wherein the discontinuous lines have a 2˜15 micron gap for every segment of 5˜100 micron.

19. The method according to claim 11, wherein the substrate can further include a nerve cell cultivation area.

20. The method according to claim 11, wherein the path-finding molecules include protein, peptide, neurotransmitter, biocompatible composite material or their mixtures thereof.

21. A method for culturing neurons using a device for axon or neurite growth according to claim 1, comprising the steps of:
providing a neuron to be cultured;
placing the neuron on a path-finding molecules configured on the device for axon or neurite growth; and
placing the device in an environment suitable for neuronal growth to undergo the growth of axon or neurite.

22. The method according to claim 21, wherein the distribution of the path-finding molecules has a pattern of discontinuous lines.

23. The method according to claim 21, wherein the distribution of the path-finding molecules has a pattern of discontinuous blocks.

24. The method according to claim 22, wherein the discontinuous lines are 2˜10 micron wide.

25. The method according to claim 22, wherein the discontinuous lines have a 2˜15 micron gap for every segment of 5˜100 micron.

26. The method according to claim 21, wherein the path-finding molecules include protein, peptide, neurotransmitter, biocompatible composite material or their mixtures thereof.
